(19) United States
(12) Patent Application Publication
(10) Pub. No.: US 2024/0181275 A1
(43) Pub. Date: Jun. 6, 2024

(54) MONTE CARLO SIMULATION-BASED PARALLEL DOSE CALCULATION METHOD, COMPUTER DEVICE, AND STORAGE MEDIUM

(71) Applicant: OUR UNITED CORPORATION, Xi'an (CN)

(72) Inventors: Bo LI, Xi'an (CN); Hong CHENG, Xi'an (CN); Yipeng JING, Xi'an (CN)

(21) Appl. No.: 18/498,346

(22) Filed: Oct. 31, 2023

(30) Foreign Application Priority Data
Dec. 2, 2022 (CN) 202211544762.9

(51) Int. Cl. A61N 5/10; G06T 1/20; G06T 1/60

(52) U.S. Cl. CPC A61N 5/1031; G06T 1/20; G06T 1/60; A61N 2005/1034

(57) ABSTRACT

Provided is a Monte Carlo simulation-based parallel dose calculation method, a computer device, and a storage medium. The method includes: acquiring sampled particles by sampling according to a radiation source model of a device, wherein information of the sampled particles includes positions, movement directions, energies, and weights of the sampled particles; stochastically generating a plurality of new positions in a region of interest along the movement directions of the sampled particles; and simulating transport processes of particles in parallel at the plurality of new positions using same particle information and same trajectory data, wherein the particle information used at the plurality of new positions is the information of the sampled particles.

### CROSS-REFERENCE TO RELATED APPLICATION

This application is based on and claims priority to Chinese Patent Application No. 202211544762.9 filed on Dec. 2, 2022, and entitled “MONTE CARLO PARALLEL DOSE CALCULATION METHOD,” and the disclosure of which is herein incorporated by reference in its entirety.

### TECHNICAL FIELD

The present disclosure relates to the field of display technologies, and in particular, relates to a Monte Carlo simulation-based parallel dose calculation method, a computer device, and a storage medium.

### BACKGROUND

A Monte Carlo simulation-based dose calculation method is a statistical simulation method based on stochastically sampling. For transport of particles, pseudo-random numbers generated by a computer are used to stochastically sample relevant physical parameters in physical transport processes of particles in a specific probability distribution in the method, such that the transport process of the single particle in a medium is simulated. The Monte Carlo simulation-based method, as the most accurate calculation method in the field of radiotherapy dose calculation, plays an important role in the fields of medical physics and radiation dosimetry due to the accuracy in particle transport processes and geometric modeling.

However, general-purpose Monte Carlo simulation tools, such as EGS4, EGSnrc, GEANT, MCNP, and the like use accurate transport algorithms and reaction section data to simulate the transport processes of the particles in different energy ranges in various mediums, but a long calculation period is a major obstacle for clinical applications. Over the past decades, for improvement of a computing efficiency and reduction of computing period, people have proposed several fast Monte Carlo computing models based on different principles, such that the computing speed of the Monte Carlo simulation-based method is improved, but there is still a lot of space for development for meeting requirements of near-real-time computing in the clinical application.

### SUMMARY

Embodiments of the present disclosure provide a Monte Carlo simulation-based parallel dose calculation method, a computer device, and a storage medium. The technical solutions are as follows.

In some embodiments of the present disclosure, a Monte Carlo simulation-based parallel dose calculation method is provided. The display panel includes:
acquiring sampled particles by sampling according to a radiation source model of a device, wherein information of the sampled particles includes positions, movement directions, energies, and weights of the sampled particles;
stochastically generating a plurality of new positions in a region of interest along the movement directions of the sampled particles; and
simulating transport processes of particles in parallel at the plurality of new positions using same particle information and same trajectory data,
wherein the particle information used at the plurality of new positions is the information of the sampled particles.

In some embodiments, simulating the transport processes of the particles in parallel at the plurality of new positions using the same particle information and the same trajectory data includes:
simulating the transport processes of the particles in parallel at the plurality of new positions by a plurality of compute unified device architecture (CUDA) threads of a graphic processing unit (GPU) using the same particle information and the same trajectory data.

In some embodiments, simulating the transport processes of the particles in parallel at the plurality of new positions by the plurality of CUDA threads of the GPU using the same particle information and the same trajectory data includes:
reading the particle information;
acquiring trajectory data corresponding to energies of the particles by any one of the plurality of CUDA threads, and writing the trajectory data to a shared memory of the GPU; and
simultaneously accessing the trajectory data in the shared memory of the GPU by the plurality of CUDA threads, and simulating the transport processes of the particles in parallel at the plurality of new positions using the particle information and the trajectory data.

In some embodiments, simulating the transport processes of the particles in parallel at the plurality of new positions by the plurality of CUDA threads of the GPU using the same particle information and the same trajectory data includes:
reading the particle information;
synergistically acquiring trajectory data corresponding to energies of the particles by at least two of the plurality of CUDA threads, and writing the trajectory data to a shared memory of the GPU; and
simultaneously accessing the trajectory data in the shared memory of the GPU by the plurality of CUDA threads, and simulating the transport processes of the particles in parallel at the plurality of new positions using the particle information and the trajectory data.

In some embodiments, the trajectory data is pre-generated trajectory data.

In some embodiments, the pre-generated trajectory data is generated by a conventional Monte Carlo simulation tool.

In some embodiments, acquiring the trajectory data corresponding to the energies of the particles by any one of the plurality of CUDA threads includes:
querying an index table of the trajectory data based on the energies of the particles by the any one of the plurality of CUDA threads, and determining an address of the trajectory data in a global memory of the GPU; and
acquiring, based on the address, the trajectory data corresponding to the energies of the particles from the global memory of the GPU by the any one of the plurality of CUDA threads.

In some embodiments, synergistically acquiring the trajectory data corresponding to the energies of the particles by the at least two of the plurality of CUDA threads, and writing the trajectory data to the shared memory of the GPU include:
querying an index table of the trajectory data based on the energies of the particles by any one of the at least two of the plurality of CUDA threads, and determining an address of the trajectory data in a global memory of the GPU; and
synergistically acquiring, based on the address, the trajectory data corresponding to the energies of the particles from the global memory of the GPU by the at least two of the plurality of CUDA threads.

In some embodiments of the present disclosure, a computer device is provided. The computer device includes one or more processors, a memory, and one or more application programs stored in the memory, wherein the one or more processors, when loading and running the one or more application programs, are caused to perform the Monte Carlo simulation-based parallel dose calculation method in the above embodiments.

In some embodiments of the present disclosure, a non-transitory computer-readable storage medium is provided. The non-transitory computer-readable storage medium stores one or more computer programs thereon, wherein the one or more computer programs, when loaded and run by a processor, cause the processor to perform the Monte Carlo simulation-based parallel dose calculation method in the above embodiments.

### BRIEF DESCRIPTION OF DRAWINGS

For clearer description of the technical solutions in the embodiments of the present disclosure, the following briefly describes the accompanying drawings required for describing the embodiments. Apparently, the accompanying drawings in the following description show merely some embodiments of the present disclosure, and those of ordinary skill in the art may still derive other drawings from these accompanying drawings without any creative efforts.

FIG. 1 is a flowchart of a Monte Carlo simulation-based dose calculation method according to some embodiments of the present disclosure;

FIG. 2 is a schematic diagram of a process of stochastically generating a plurality of new positions according to some embodiments of the present disclosure;

FIG. 3 is a schematic diagram of a method of simulating transport processes of particles in parallel by a plurality of compute unified device architecture (CUDA) threads of a graphic processing unit (GPU) according to some embodiments of the present disclosure;

FIG. 4 is a schematic diagram of another method of simulating transport processes of particles in parallel by a plurality of CUDA threads of a GPU according to some embodiments of the present disclosure; and

FIG. 5 is a schematic structural diagram of a computer device according to some embodiments of the present disclosure.

### DETAILED DESCRIPTION

The technical solutions in the embodiments of the present disclosure are described clearly and completely in conjunction with the accompanying drawings in the embodiments of the present disclosure hereinafter. Apparently, all other embodiments derived based on the embodiments of the present disclosure by those skilled in the art without creative efforts shall fall within the protection scope of the present disclosure.

In the embodiments of the present disclosure, unless otherwise defined, the term “a plurality of” herein means two or more. In the present disclosure, the term “illustratively” is used to represent “for example, for illustration, or for description.” Any embodiment described by the term “illustratively” in the present disclosure is not necessarily interpreted as being preferable or superior to other embodiments. The following description is shown to enable those skilled in the art to implement and use the present disclosure. In the following description, details are given for the purposes of explanation. It should be understood that a person of ordinary skill in the art may recognize that the present disclosure can be implemented without the use of these particular details. In other embodiments, common structures and processes will not be elaborated in order to avoid that the description of the present disclosure is obscure due to unnecessary details. Accordingly, the present disclosure is not intended to be limited to the shown embodiments, but to be consistent with the broadest range of principles and features disclosed in the present disclosure.

It should be noted that as the method in the embodiments of the present disclosure is performed in the computer device, processing objects in the computer device are described in a form of data or information. For example, time is actually time information. It should be noted that sizes, numbers, positions, and the like in the following embodiments are described in the form of corresponding data to facilitate being processed on the computer device, which are not repeated herein.

Embodiments of the present disclosure provide a Monte Carlo simulation-based parallel dose calculation method, a computer device, and a storage medium, and the Monte Carlo simulation-based parallel dose calculation method includes a process of simulating transport processes of particles in parallel using same trajectory data of the particles, such that the speed of the Monte Carlo simulation-based dose calculation is improved.

The Monte Carlo simulation-based parallel dose calculation method in the embodiments of the present disclosure is applicable to the field of radiotherapy, and the method is used to simulate transport processes of particles and calculate the dose distribution in the treatment plan in the process of making the treatment plan to verify the quality of the treatment plan. The embodiments of the present disclosure first provide a Monte Carlo simulation-based dose calculation method, and the performing object of the Monte Carlo simulation-based dose calculation method is a processor in the computer device. The Monte Carlo simulation-based dose calculation method includes:
acquiring sampled particles by sampling according to a radiation source model of a device, wherein information of the sampled particles includes positions, movement directions, energies, and weights of the sampled particles; stochastically generating a plurality of new positions in a region of interest along the movement directions of the sampled particles; and simulating transport processes of particles in parallel at the plurality of new positions using same particle information and same trajectory data, wherein the particle information used at the plurality of new positions is the information of the sampled particles.

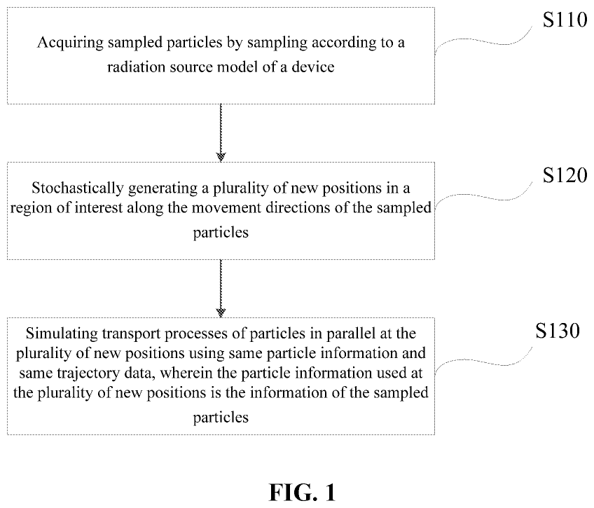

FIG. 1 is a flowchart of a Monte Carlo simulation-based dose calculation method according to some embodiments of the present disclosure. As shown in FIG. 1, the Monte Carlo simulation-based dose calculation method includes the following S110 to S130.

In S110, sampled particles are acquired by sampling according to a radiation source model of a device.

In the present disclosure, a processor in a computer device acquires the sampled particles by sampling according to the radiation source model of a radiotherapy device. The information of the sampled particles includes positions, movement directions, energies, and weights of the sampled particles.

In the embodiments of the present disclosure, the radiation source model of the radiotherapy device is established for the Monte Carlo simulation-based dose calculation method with reference to the structure of the radiotherapy device, and the sampled particles are then acquired by sampling according to the radiation source model. The sampling method used in the sampling operation includes a cross-correlation sampling method, a direct sampling method, an acceptance and rejection sampling method, an importance sampling method, and the like.

The information of the sampled particles acquired by sampling includes position information, movement direction information, energy information, and weight information of the sampled particles, and the like. Generally, various sampled particles are acquired by the sampling operation, and the sampled particles are differed in the position, the energy, the movement direction, and the weight due to the stochastic sampling processes.

In S120, a plurality of new positions are stochastically generated in a region of interest along the movement directions of the sampled particles.

In the present disclosure, the plurality of new positions are stochastically generated in the region of interest along the movement directions of the sampled particles upon the sampling operation of the processor in the computer device.

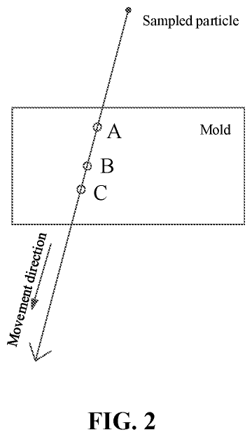

Illustratively, as shown in FIG. 2, FIG. 2 is a schematic diagram of a process of stochastically generating a plurality of new positions according to some embodiments of the present disclosure. The sampled particles enter from an outer of a mold to an inner of the mold along the movement directions of the sampled particles, and a plurality of new positions (that is, the position A, the position B, and the position C) are stochastically generated in the inner of the mold along the movement directions of the sampled particles. In the embodiments of the present disclosure, distances between the plurality of stochastically generated new positions are equal or not equal. That is, distances among the position A, the position B, and the position C are equal or not equal.

In the embodiments of the present disclosure, the region of interest generally refers to an inner region of the mold used in the Monte Carlo simulation. Generally, the mold used in the Monte Carlo simulation includes a standard water mold.

In S130, transport processes of particles are simulated in parallel at the plurality of new positions using same particle information and same trajectory data.

In the present disclosure, the processor in the computer device simulates the transport processes of the particles in parallel at the plurality of new positions using the same particle information and the same trajectory data. The particle information used at the plurality of new positions is the information of the sampled particles acquired by sampling in S110.

Generally, a number of successive reactions occur between the particles and the medium during transport and data, such as the reaction type, the energy loss, the energy deposition, the transport path length, and the scattering direction in each reaction is recorded in a form of trajectory data. Thus, in the embodiments of the present disclosure, the trajectory data includes the data, such as the reaction type, the energy loss, the energy deposition, the transport path length, and the scattering direction in each reaction between the particles and the medium in the transport process.

In the embodiments of the present disclosure, the trajectory data can be computationally generated in real time during the Monte Carlo simulation process. For example, the trajectory data of the generated particles is calculated in real time upon the sampling process and is stored (for example, stored in a global memory of a central processing unit (CPU/a graphic processing unit (GPU)) for the convenient use in the subsequent processes. In some embodiments, the trajectory data is pre-generated prior to the Monte Carlo simulation process, that is, the trajectory data is pre-generated trajectory data. The pre-generated trajectory data is accurately generated by a conventional Monte Carlo simulation tool, for example, GEANT4, EGS4/5, EGSnrc, MCNP, PENELOPE, and the like, and the pre-generated trajectory data is stored for use in the subsequent processes.

As transport behaviors of the particles with less energy differences meet a specific physics rule when a physical reaction occurs between the particles and the medium, the various particles are classified in different energy levels according to the energies. Transport processes of the particles in the same energy level are simulated, and trajectory data is recorded as trajectory data of all particles in the energy level.

In the embodiments of the present disclosure, assuming that the sampled particles in the mold at the new positions are reacted in the same way with the medium at the same time, the simulation on the transport process of one sampled particle is equivalent to simulation on transport processes of multiple particles with less differences in particle information, such that an efficiency of the Monte Carlo simulation is improved. Meanwhile, the parallel simulation is performed on the transport processes of the particles at the plurality of new positions in the embodiments of the present disclosure, such that the simulation speed and the simulation efficiency are further improved.

In the embodiments of the present disclosure, the process of simulating the transport processes of the particles in parallel at the plurality of new positions using the same particle information and the same trajectory data are performed based on the CPU. For example, the parallel simulation is performed based on the CPU by a parallel simulation frame, such as multi-threads, multi-processes, or OpenMP, MPI, and the like. Alternatively, the parallel simulation is performed based on a GPU, for example, a plurality of compute unified device architecture (CUDA) threads of the GPU.

Generally, when a CUDA threads of the GPU are running, all the threads are rearranged into a number of BLOCKs, each BLOCK contains multiple WARPs, and each WARP contains 32 threads, so the number of threads within each BLOCK is a multiple of 32. Meanwhile, the shared memory of the GPU can be accessed in a shared way among all threads within a BLOCK.

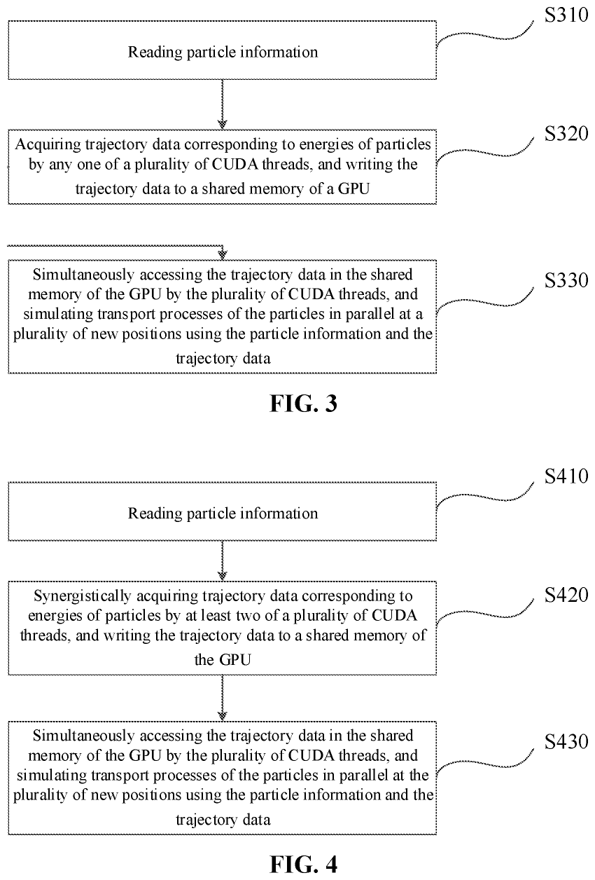

Thus, in the process of simulating the transport processes of the particles in parallel by the plurality of CUDA threads of the GPU, the parallel simulation is performed by a plurality of CUDA threads belonging to the same WARP of the GPU in some embodiments, and the parallel simulation is performed by a plurality of CUDA threads in different WARPs belonging to the same BLOCK of the GPU in some embodiments. FIG. 3 is a schematic diagram of a method of simulating transport processes of particles in parallel by a plurality of compute unified device architecture (CUDA) threads of a graphic processing unit (GPU) according to some embodiments of the present disclosure. As shown in FIG. 3, the method includes S310 to S330.

In S310, particle information is read.

Prior to the parallel simulation on the transport processes of the particles, the plurality of CUDA threads of the GPU require to acquire the information of the to-be-simulated particles from the global memory of the GPU, and the information of the particles generally includes the position information, the movement direction information, the energy information, and the weight information of the particles.

In S320, trajectory data corresponding to energies of the particles is acquired by any one of the plurality of CUDA threads, and the trajectory data is written to a shared memory of the GPU.

Upon acquisition of the information of the particles, any one of the plurality of CUDA threads of the GPU acquires the trajectory data corresponding to the energy information (or energy level information) of the particles from the shared memory of the GPU based on the acquired energy information of the particles. In addition, the acquired trajectory data is written to the shared memory of the GPU, such that the trajectory data is shared and accessible by all of the plurality of CUDA threads of the GPU.

It should be noted that as a space of the shared memory is limited, the trajectory data cannot be completely saved in the shared memory in the case that the trajectory data requires a great memory space. In actual practices, such case is processed in a lazy load manner. Specifically, the currently desired trajectory data is loaded in the shared memory, the transport processes of the particles are simulated based on the currently desired trajectory data, and the occupied space of the shared memory is released in the case that the data is not used in the subsequent processes to load required remaining trajectory data.

In S330, the trajectory data in the shared memory of the GPU is simultaneously accessed by the plurality of CUDA threads, and the transport processes of the particles are simulated in parallel at the plurality of new positions using the particle information and the trajectory data.

Upon acquisition of the trajectory data, the plurality of CUDA threads of the GPU simultaneously access the trajectory data in the shared memory of the GPU, and simulate the transport processes of the particles in parallel at the plurality of new positions using the same particle information and the same trajectory data.

The plurality of CUDA threads of the GPU simultaneously access the same address at the same time in accessing the shared memory, the GPU hardware performs a broadcast access, and thus no bank conflict exists. As the access speed of the shared memory is greatly greater than the access speed of the global memory, the access efficiency of the memory is greatly improved.

In the embodiments of the present disclosure, as the transport processes of the particles are simulated using the same particle information and the same trajectory data, the thread differentiation does not occur in the execution process of the plurality of CUDA threads of the GPU, such that the parallel execution efficiency of the CUDA threads is improved, and the calculation efficiency of the Monte Carlo simulation-based method is improved. Meanwhile, the trajectory data is acquired by any one of the plurality of CUDA threads and is stored in the shared memory of the GPU, and all of the plurality of CUDA threads simultaneously access the shared memory of the GPU to acquire the trajectory data and simulate the transport processes of the particles, such that all of the plurality of CUDA threads do not need to individually and decentralized access the global memory of the GPU, the access efficiency of the memory by the plurality of CUDA threads of the GPU is improved, and the calculation efficiency of the Monte Carlo simulation-based method is improved.

FIG. 4 is a schematic diagram of another method of simulating transport processes of particles in parallel by a plurality of CUDA threads of a GPU according to some embodiments of the present disclosure. As shown in FIG. 4, the method includes S410 to S430.

In S410, particle information is read.

It should be noted that S410 in the embodiments is the same as S310 in the above embodiments of FIG. 3, which is not repeated herein.

In S420, trajectory data corresponding to energies of the particles is synergistically acquired by at least two of the plurality of CUDA threads, and the trajectory data is written to a shared memory of the GPU.

In the embodiments of the present disclosure, synergistically acquiring the trajectory data by the at least two of the plurality of CUDA threads includes that each of the at least two of the plurality of CUDA threads respectively acquire different portions of the trajectory data, and the trajectory data acquired by the at least two of the plurality of CUDA threads compose the complete trajectory data. For example, the complete trajectory data is arranged into two portions, and the two portions are synergistically acquired by two of the plurality of CUDA threads. That is, a first CUDA thread acquires a first portion of the trajectory data, and a second CUDA thread acquires a second portion of the trajectory data, such that the complete trajectory data is synergistically acquired by the two CUDA threads.

In the embodiments of the present disclosure, in synergistically acquiring the trajectory data by at least two CUDA threads, the trajectory data is acquired in an order (in sequence) or in parallel. The duration of acquiring the trajectory data is shortened and the acquiring efficiency is improved by acquiring the trajectory data in parallel by the at least two CUDA threads.

In S430, the trajectory data in the shared memory of the GPU is simultaneously accessed by the plurality of CUDA threads, and the transport processes of the particles are simulated in parallel at the plurality of new positions using the particle information and the trajectory data.

It should be noted that S430 in the embodiments is the same as S330 in the above embodiments of FIG. 3, which is not repeated herein.

In the embodiments of the present disclosure, it can be time-consuming in acquiring the trajectory data by accessing the global memory of the GPU by the CUDA threads of the GPU and determining the position of the trajectory data in the global memory in an order, and thus the access delay may exist. For reduction of the access delay of the global memory, an index table of the trajectory data is queried to determine the position of the trajectory data in the global memory to acquire the trajectory data.

In the embodiments of the present disclosure, the index table of the trajectory data is established according to a corresponding relationship of the energies (or the energy level) of the particles and the stored position of the trajectory data in the global memory of the GPU. In the case that the plurality of CUDA threads of the GPU acquire the trajectory data, the CUDA threads determine the address of the trajectory data corresponding to the energies of the particles in the global memory of the GPU by querying the index table of the trajectory data based on the energies of the particles, and then acquire the corresponding trajectory data from the global memory of the GPU based on the address.

Illustratively, acquiring the trajectory data corresponding to the energies of the particles by any one of the plurality of CUDA threads includes:
querying an index table of the trajectory data based on the energies of the particles by the any one of the plurality of CUDA threads, and determining an address of the trajectory data corresponding to the energies of the particles in a global memory of the GPU; and
acquiring, based on the acquired address of the trajectory data, the trajectory data corresponding to the energies of the particles from the global memory of the GPU by the any one of the plurality of CUDA threads.

In some embodiments, the thread for determining the address of the trajectory data and the thread for acquiring the trajectory data are the same thread or different threads.

Illustratively, synergistically acquiring trajectory data corresponding to energies of the particles by the at least two of the plurality of CUDA threads, and writing the trajectory data to a shared memory of the GPU include:
querying an index table of the trajectory data based on the energies of the particles by any one of the plurality of CUDA threads, and determining an address of the trajectory data corresponding to the energies of the particles in a global memory of the GPU; and
synergistically acquiring, based on the address of the trajectory data, the trajectory data corresponding to the energies of the particles from the global memory of the GPU by the at least two of the plurality of CUDA threads.

The any thread for determining the trajectory data is the thread in the at least two of the plurality of CUDA threads for synergistically acquiring the trajectory data or the thread in the plurality of CUDA threads different from the at least two of the plurality of CUDA threads for synergistically acquiring the trajectory data.

In the embodiments of the present disclosure, the transport processes of particles are simulated in parallel using the same particle information and the same trajectory data, such that the calculation efficiency of the Monte Carlo simulation-based method is improved, and the duration of calculating the dose is reduced.

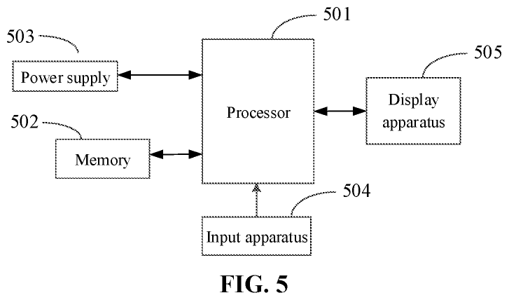

The embodiments of the present disclosure further provide a computer device. The computer device includes one or more processors, a memory, and one or more application programs stored in the memory. The one or more processors, when loading and running the one or more application programs, are caused to perform the processes in the Monte Carlo simulation-based parallel dose calculation method in any above embodiment. The embodiments of the present disclosure further provide a computer device, as shown in FIG. 5, FIG. 5 is a schematic structural diagram of a computer device according to some embodiments of the present disclosure.

The computer device includes assemblies, such as a processor 501 having one or more processing cores, a memory 502 of one or more computer-readable storage medias, a power supply 503, and an input apparatus 504. It can be understood by those skilled in the art that the structure of the computer device shown in FIG. 5 does not constitute a limitation to the computer device.

The computer device includes more or fewer assemblies than those shown in the drawing, or combines some components, or uses different component arrangements.

The processor 501 is a control center of the computer device, and connects various parts of the entire computer device through various interfaces and lines. By running or executing software programs and/or modules stored in the memory 502 and invoking data stored in the memory 502, various functions and processing data of the computer device are implemented, such that the computer device is monitored.

In some embodiments, the processor 501 includes one or more processing cores. In some embodiments, the processor 501 is integrated as an application processor and a modulation and demodulation processor. The application processor mainly processes an operating system, a user interface, an application, and the like, and the modulation and demodulation processor mainly processes wireless communication. It can be understood that the above modulation and demodulation processor may also not be integrated into the processor 501.

The memory 502 is configured to store software programs and modules, and the processor 501 runs various functional applications and data processing by running the software programs and the modules stored in the memory 502. The memory 502 mainly includes a program storage region and a data storage region. The program storage region stores the operating system, an application required for at least one function (such as a sound playing function and an image playing function), and the like. The data storage region stores data created based on the use of the computer device, and the like. In addition, the memory 502 includes a high-speed random access memory and a non-volatile memory, such as at least one of a disk storage device, a flash memory device, or other volatile solid-state storage devices. Correspondingly, the memory 502 also includes a memory controller to provide access to the memory 502 by the processor 501.

The computer device also includes the power supply 503 that supplies power to various assemblies. In some embodiments, the power supply 503 is logically connected to the processor 501 through a power supply management system, such that the functions, such as management of charging, discharging, and power consumption are achieved through the power supply management system. The power supply 503 further includes one or more direct current or alternating current power supplies, a recharging systems, a power failure detection circuit, a power converter or an inverter, a power status indicator, and any other assemblies.

The computer device also includes an input apparatus 504. The input apparatus 504 is configured to receive input digital or character information, and generate keyboard, mouse, joystick, optical, or trackball signal input related to user settings and function control.

Although not shown, the computer device further includes a display apparatus 505. The display apparatus 505 is a display, and thus is not repeated herein. Specifically, in the embodiments, the processor 501 in the computer device loads, according to the following instructions, executable files corresponding to one or more programs into the memory 502, and the processor 501 runs the programs stored in the memory 502 to:
acquire sampled particles by sampling according to a radiation source model of a device, wherein information of the sampled particles includes positions, movement directions, energies, and weights of the sampled particles;
stochastically generate a plurality of new positions in a region of interest along the movement directions of the sampled particles; and
simulate transport processes of particles in parallel at the plurality of new positions using same particle information and same trajectory data,
wherein the particle information used at the plurality of new positions is the information of the sampled particles.

Those of ordinary skill in the art may understand that all or part of the processes in the various methods of the above embodiments can be performed by instructions, or by controlling related hardware through instructions. The instructions may be stored in a computer-readable storage medium, and loaded and executed by the processor.

For this purpose, some embodiments of the present disclosure provide a non-transitory computer-readable storage medium. The storage medium includes a read-only memory (ROM), a random access memory (RAM), a disk or an optical disc, and the like. Computer programs are stored on the storage medium, and the computer programs, when loaded and run by a processor, cause the processor to perform any Monte Carlo simulation-based parallel dose calculation method in the embodiments of the present disclosure. For example, the computer programs, when loaded and run by a processor, cause the processor to:
acquire sampled particles by sampling according to a radiation source model of a device, wherein information of the sampled particles includes positions, movement directions, energies, and weights of the sampled particles;
stochastically generate a plurality of new positions in a region of interest along the movement directions of the sampled particles; and
simulate transport processes of particles in parallel at the plurality of new positions using same particle information and same trajectory data,
wherein the particle information used at the plurality of new positions is the information of the sampled particles.

In the above embodiments, the description of each embodiment has its own emphasis. The parts not detailed in an embodiment may refer to the detailed description for other above embodiments and are not repeated herein.

In specific implementations, the above various structures are implemented as independent entities, or are arbitrarily combined to be implemented as the same or several entities. The specific implementations of the above various structures may refer to the above method embodiments, and thus are not repeated herein.

The specific implementations of the above various operations may refer to the above embodiments, and thud are not repeated herein.

The Monte Carlo simulation-based parallel dose calculation method, the computer device, and the storage medium in the embodiments of the present disclosure are described in detail herein. Specific examples are used herein to explain the principles and embodiments of the present disclosure. The descriptions of the above embodiments are only used to help understand the method and the core idea of the present disclosure. Meanwhile, those skilled in the art will be changed in the specific embodiments and the application scope according to the idea of the present disclosure. In summary, the content of the Specification should not be understood as a limitation to the present disclosure.

1. A Monte Carlo simulation-based parallel dose calculation method, performed by a computer device, comprising:
acquiring sampled particles by sampling according to a radiation source model of a device, wherein information of the sampled particles comprises positions, movement directions, energies, and weights of the sampled particles;
stochastically generating a plurality of new positions in a region of interest along the movement directions of the sampled particles; and
simulating transport processes of particles in parallel at the plurality of new positions using same particle information and same trajectory data, wherein the particle information used at the plurality of new positions is the information of the sampled particles.

2. The method according to claim 1, wherein simulating the transport processes of the particles in parallel at the plurality of new positions using the same particle information and the same trajectory data comprises:
simulating the transport processes of the particles in parallel at the plurality of new positions by a plurality of compute unified device architecture (CUDA) threads of a graphic processing unit (GPU) using the same particle information and the same trajectory data.

3. The method according to claim 2, wherein simulating the transport processes of the particles in parallel at the plurality of new positions by the plurality of CUDA threads of the GPU using the same particle information and the same trajectory data comprises:
reading the particle information;
acquiring trajectory data corresponding to energies of the particles by any one of the plurality of CUDA threads, and writing the trajectory data to a shared memory of the GPU; and
simultaneously accessing the trajectory data in the shared memory of the GPU by the plurality of CUDA threads, and simulating the transport processes of the particles in parallel at the plurality of new positions using the particle information and the trajectory data.

4. The method according to claim 2, wherein simulating the transport processes of the particles in parallel at the plurality of new positions by the plurality of CUDA threads of the GPU using the same particle information and the same trajectory data comprises:
reading the particle information;
synergistically acquiring trajectory data corresponding to energies of the particles by at least two of the plurality of CUDA threads, and writing the trajectory data to a shared memory of the GPU; and
simultaneously accessing the trajectory data in the shared memory of the GPU by the plurality of CUDA threads, and simulating the transport processes of the particles in parallel at the plurality of new positions using the particle information and the trajectory data.

5. The method according to claim 1, wherein the trajectory data is pre-generated trajectory data.

6. The method according to claim 5, wherein the pre-generated trajectory data is generated by a conventional Monte Carlo simulation tool.

7. The method according to claim 3, wherein acquiring the trajectory data corresponding to the energies of the particles by any one of the plurality of CUDA threads comprises:
querying an index table of the trajectory data based on the energies of the particles by the any one of the plurality of CUDA threads, and determining an address of the trajectory data in a global memory of the GPU; and
acquiring, based on the address, the trajectory data corresponding to the energies of the particles from the global memory of the GPU by the any one of the plurality of CUDA threads.

8. The method according to claim 4, wherein synergistically acquiring the trajectory data corresponding to the energies of the particles by the at least two of the plurality of CUDA threads, and writing the trajectory data to the shared memory of the GPU comprise:
querying an index table of the trajectory data based on the energies of the particles by any one of the at least two of the plurality of CUDA threads, and determining an address of the trajectory data in a global memory of the GPU; and
synergistically acquiring, based on the address, the trajectory data corresponding to the energies of the particles from the global memory of the GPU by the at least two of the plurality of CUDA threads.

9. A computer device, comprising: one or more processors, a memory, and one or more application programs stored in the memory, wherein the one or more processors, when loading and running the one or more application programs, are caused to:
acquire sampled particles by sampling according to a radiation source model of a device, wherein information of the sampled particles comprises positions, movement directions, energies, and weights of the sampled particles;
stochastically generate a plurality of new positions in a region of interest along the movement directions of the sampled particles; and
simulate transport processes of particles in parallel at the plurality of new positions using same particle information and same trajectory data, wherein the particle information used at the plurality of new positions is the information of the sampled particles.

10. A non-transitory computer-readable storage medium, storing one or more computer programs thereon, wherein the one or more computer programs, when loaded and run by a processor, cause the processor to:
acquire sampled particles by sampling according to a radiation source model of a device, wherein information of the sampled particles comprises positions, movement directions, energies, and weights of the sampled particles;
stochastically generate a plurality of new positions in a region of interest along the movement directions of the sampled particles; and
simulate transport processes of particles in parallel at the plurality of new positions using same particle information and same trajectory data, wherein the particle information used at the plurality of new positions is the information of the sampled particles.
